(19) United States
(12) Patent Application Publication
(10) Pub. No.: US 2005/0074759 A1
(43) Pub. Date: Apr. 7, 2005

(54) Methods for evaluation of in vitro aminoacyl tRNA production using matrix-assisted laser desorption/ionization time-of-flight mass spectrometry

(76) Inventors: E. James Petersson, Pasadena, CA (US); Mona Shahgholi, Pasadena, CA (US); Henry A. Lester, Pasadena, CA (US); Dennis A. Dougherty, South Pasadena, CA (US)

Correspondence Address: Toni-Junell Herbert;Reed Smith LLP, 1301 K Street, N.W., Suite 1100 - East Tower, Washington, DC, 20005-3373, US

(21) Appl. No.: 10/420,967

(22) Filed: Apr. 23, 2003

Related U.S. Application Data
(60) Provisional application No. 60/375,162, filed on Apr. 24, 2002.
(60) Provisional application No. 60/374,809, filed on Apr. 24, 2002.

(51) Int. Cl. C12Q 1/68

(52) U.S. Cl. 435/6

(57) ABSTRACT

The present invention relates to a new use for matrix-assisted laser desorption/ionization time-of-flight mass spectrometry (MALDI-TOF MS). Specifically, the present invention provides a new method for evaluating the aminoacylation state of tRNAs, which are useful for site-directed mutagenesis of both sidechain and backbone protein structures. Further, the present invention useful for the generation of novel peptides, polypeptides, and proteins that may be used in drug design and screening, and in the design of small molecule agonists or antagonists for receptors, enzymes and other proteins and molecules involved in various disease states. Additionally, the present invention is useful in the field of tRNA aminoacylation, particularly in the design of synthetases.

This application claims priority to Provisional Application Ser. No. 60/375,162; Filed Apr. 23, 2002 and Provisional Application Ser. No. 60/374,809; Filed: Apr. 24, 2002.

The development of the present invention was funded in part by the National Institutes of Health (NS-34407 and NS-11756), hence, the United States Government may have certain rights relating to this patent application.

### I. FIELD OF THE INVENTION

The present invention discloses a new use for matrix-assisted laser desorption/ionization time-of-flight mass spectrometry (MALDI-TOF MS). Specifically, the present invention provides a new method for evaluating the aminoacylation state of tRNAs, which are useful for site-directed mutagenesis of both sidechain and backbone protein structures. Further, the present invention useful for the generation of novel peptides, polypeptides, and proteins that may be used in drug design and screening, and in the design of small molecule agonists or antagonists for receptors, enzymes and other proteins and molecules involved in various disease states. Additionally, the present invention is useful in the field of tRNA aminoacylation, particularly in the design of synthetases.

### II. BACKGROUND OF THE INVENTION

Site-specific manipulation of both sidechain and backbone protein structures can be achieved by nonsense suppression incorporation of unnatural amino acids into the protein structures. See, Cornish V W, Mendel D, Schultz P G. (1995), Angew Chem Int Ed Engl 34:621-633; and, Dougherty D A. (2000), Curr Opin Chem Biol 4:645-652. Using both in vitro and in vivo expression systems, well over a hundred unnatural amino acids have been incorporated into dozens of different proteins. This methodology requires the in vitro production of an aminoacyl suppressor tRNA, which is then used to deliver an unnatural amino acid at the site of a mutagenically introduced stop codon. Aminoacyl suppressor tRNAs are made by ligating a chemically synthesized aminoacyl dCA dinucleotide to the 3′ end of a transcribed 74mer tRNA.

There is no simple or facile technique for evaluating the aminoacylation state of a suppressor tRNA prior to using it to deliver an unnatural amino acid at the site of a mutagenically introduced stop codon. Current methods include the use of gel electrophoresis, which infers mass from electrophoretic mobility that is slower and less material efficient than MALDI-TOF MS analysis, or radiolabelling, which is too laborious and hence impractical for everyday production of tRNAs for suppression (Weygand-Durasevic I, Lenhard D, Filipic S, Soll D. (1996), J Biol Chem 271:2455-2461).

Moreover, standard acid/urea gel electrophoresis used to evaluate tRNA aminoacylation, requires long running times of approximately 36 hours (Kohrer C, Xie L, Kellerer S, Varshney U, Rajbhandary U L. (2001), Proc Natl Acad Sci USA 98:14310-14315; Varshney U, Lee C P, Rajbhandary U L. (1991), J Biol Chem 266:24712-24718; Wolfson A D, Pleiss J A, Uhlenbeck O C. (1998), RNA 4:1019-1023). The long running time results in substantial amounts of the 76mer hydrolysis products being observed, because some of the 76mer aminoacylated tRNA hydrolyzes during the 36-hour process of running the gel.

The present invention provides a new use for matrix-assisted laser desorption/ionization time-of-flight mass spectrometry (MALDI-TOF MS), as described by Bakhtiar and Nelson R W. (2000), Biochem Pharmacol 59:891-905, Bakhtiar and Tse F L S. (2000), Mutagenesis 15:415-430, Kirpekar F, Douthwaite S, Roepstorff P. (2000), RNA 6:296-306, and Zenobi R, Knochenmuss R. (1998), Mass Spectrom Rev 17:337-366. The present invention provides a simple and quick way of evaluating the aminoacylation state of suppressor tRNAs that are used in site-directed mutagenesis of sidechain and backbone protein structures using MALDI-TOF MS. The present invention is much more precise than currently available techniques, thus enabling one skilled in the art to distinguish tRNA species down to a single nucleotide, and to verify the identity of amino acids.

There has been some earlier use of MALDI-TOF MS with tRNAs, for example see, Gruic-Sovulj I, et al. (1997), J Biol Chem 272:32084-32091; Rubelj et al. (1990), Eur J Biochem 193:783-788; Sochacka et al. (2000), Nucleosides Nucleotides Nucleic Acids 19:515-531; Gruic-Sovulj et al. (2001), Saccharomyces cerevisiae. Croatica Chemica Acta 74 :161-171; and, Wei & Lee (1997), Anal Chem 69:4899-4904. However, the use of MALDI-TOF MS to evaluate the production of an aminoacylated (aa) 76mer tRNA by following the disappearance of a 74mer starting material, and the appearance of a desired α-76mer is novel and has not been described previously.

A successful application of MALDI-TOF MS takes less time to run, uses less tRNA material, and is more precise than currently available techniques. More importantly, MALDI-TOF MS provides more precise aminoacylation information than can be obtained through standard gel techniques.

### III. SUMMARY OF THE INVENTION

In a preferred embodiment, the present invention comprises a method of evaluating the aminoacylated state of tRNAs using matrix-assisted laser desorption/ionization time-of-flight mass spectrometry (MALDI-TOF MS), for use in the site-directed mutagenesis of backbone and sidechain protein structures, wherein unnatural amino acids are introduced into said protein structures.

In another embodiment, the present invention discloses a method of evaluating the aminoacylated state of tRNAs using matrix-assisted laser desorption/ionization time-of-flight mass spectrometry (MALDI-TOF MS), wherein the aminoacylated tRNA is produced by a ligation reaction.

In a further embodiment, the present invention discloses a method of evaluating the aminoacylated state of tRNAs using matrix-assisted laser desorption/ionization time-of-flight mass spectrometry (MALDI-TOF MS), wherein the tRNA to be aminoacylated is a 74mer, and the aminoacylated tRNA is a 76mer.

In another embodiment, the present invention discloses a method for evaluating the aminoacylation state of a suppressor tRNA that is used to introduce unnatural amino acids into a sidechain or backbone protein by site-directed mutagenesis comprising,
(a) transcribing a desired 74-mer tRNA from a linearized cDNA, wherein said tRNA includes a 74-mer THG733;
(b) ligating the 5′ end of a protected aminoacyl dCA dinucleotide to the 3′ end of said 74mer tRNA; and,
(c) verifying the aminoacylated state of said suppressor tRNA using matrix-assisted laser desorption/ionization time-of-flight mass spectrometry (MALDI-TOF MS).

### IV. FIGURES

FIG. 1. Illustrates the T4 RNA ligase reaction. The synthesized aminoacyl dinucleotide dCA is ligated to transcribed 74mer tRNA with T4 RNA ligase to give aminoacyl 76mer tRNA. Hydrolysis of the 3′ ester bond gives 76mer tRNA. PG indicates that α-amine is protected as an amide functionality.

FIG. 2. Illustrates gel separation of the tRNA species. 20% polyacrylamide acid/urea gel showing single base resolution. 74mer and 76/77mer (in gray) are transcribed from cDNA. Corresponding markers are show in gray at left. All others (in black) are produced through T4 RNA ligase reaction of either dCA or dCA-aa with transcribed 74mer. One can observe both the α-76mer and 76mer hydrolysis product, marked at right in black.

FIG. 3. Illustrates MALDI-TOF mass spectra of various tRNA species. The tRNA species shown are the same as those shown in the gel in FIG. 2. Comparing the 74mer to the dCA-76mer shows that the addition of the dinucleotide can be clearly observed. The further increase in mass attributable to the amino acid is also clear in the Ala-76mer, Trp-76mer, and CN-Trp-76mer spectra. A small amount of 76mer hydrolysis product is apparent in the spectra of the aminoacyl tRNA. Observed masses (average of 5 spectra) are shown, with expected masses given in parentheses. The MS data confirms gel data indicating the presence of untemplated 77mer in the transcribed 76mer.

FIG. 4. Illustrates the T4 RNA ligase reaction efficiency. MALDI-TOF mass spectra of aliquots of the ligation of 5-CN-Trp-dCA taken after various reaction times. At 10 minutes, starting material (74mer), hydrolysis product (dCA 76mer) and desired product (CN-Trp 76mer) are clearly seen. The reaction is complete after 20-30 minutes, and longer reaction times lead only to increased hydrolysis (giving dCA 76mer).

### V. DETAILED DESCRIPTION

All references cited herein are hereby incorporated by reference in their entirety. Nothing herein is to be construed as an admission that the invention is not entitled to antedate such disclosure by virtue of prior invention or that the prior art provides an enabling or adequate disclosure. Throughout this description, the preferred embodiments and examples shown should be considered as exemplary, rather than as limitations on the present invention.

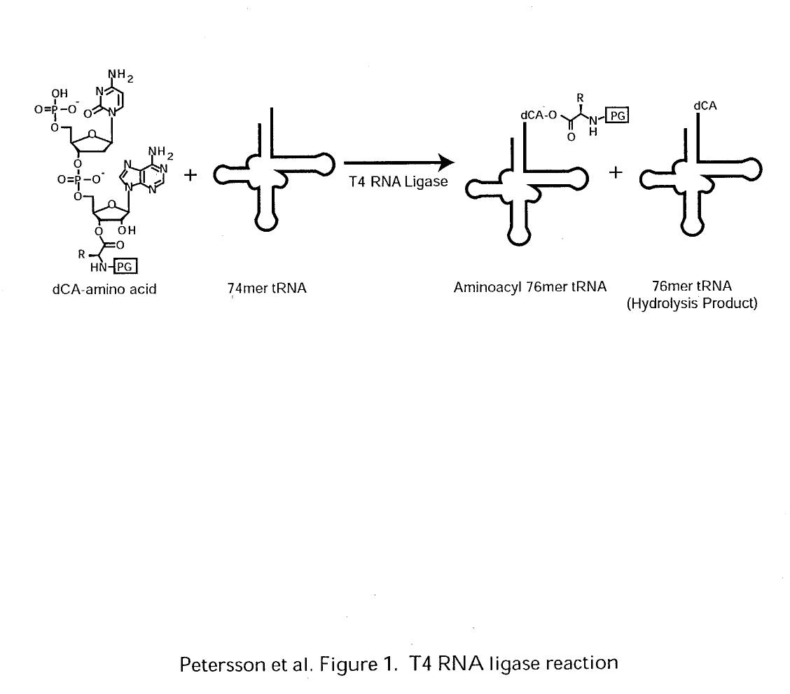

Aminoacylated suppressor tRNAs (aa-tRNA) are produced by a combination of chemical and biological steps. The desired amino acid to be delivered using such aa-tRNAs is made as an α-amino-protected cyanomethyl ester which is coupled to an artificially synthesized phospho-dCA (pdCA). A suppressor tRNA is transcribed from linearized cDNA as a 74mer lacking its 3′end CA. This transcript is ligated to the 5′ end of the pdCA-amino acid (dCA-aa) using T4 RNA ligase (FIG. 1). See also, Silber et al. (1972), Proc Natl Acad Sci USA 69:3009-3013; Uhlenbeck (1983), Trends Biochem Sci 8:94-96; and, Uhlenbeck & Gumport (1982), The Enzymes. New York, N.Y.: Academic Press, Inc. pp 31-58. The amino acid is then deprotected just prior to use in translation with an mRNA bearing the stop codon to which the tRNA will deliver its amino acid.

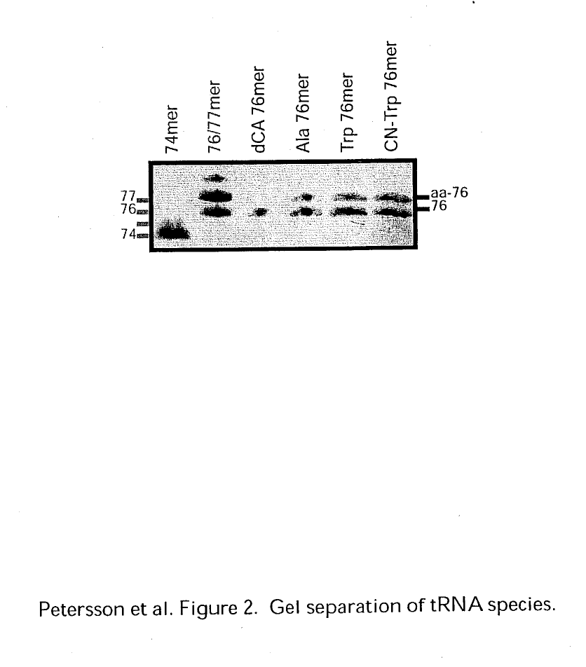

Traditionally, the reactions in the chemical steps are monitored by thin-layer chromatography, and high performance liquid chromatography. The ligation step, further, is monitored by gel electrophoresis (FIG. 2).

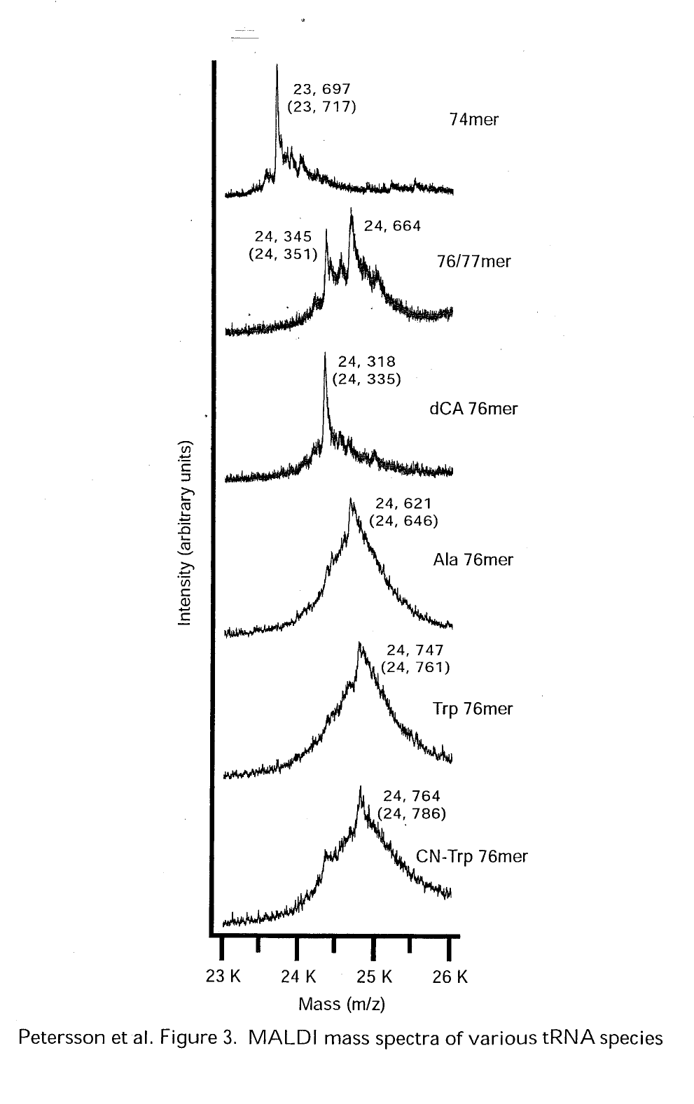

Detection of a unligated 74-mer and the desired 76-mer (for example Ala-76mer, Trp-76mer, and 5-cyano-tryptophan-76mer) using acid/urea gel electrophoresis, results in the hydrolysis of some of the desired α-76mer in the 36 hour process of running the gel, where the gel shows nearly equivalent amounts of the desired α-76mer and the 76mer hydrolysis product (FIG. 2). In contrast, mass spectra of the same tRNA samples (FIG. 3) show them to be relatively free of the 76mer hydrolysis product. This illustrates one of the several advantages of MALDI-TOF MS over gel electrophoresis as an analytical tool.

In order to identify a suitable matrix for analysis of the tRNAs, a variety of matrices may be tested with 74mer transcripts. These include 3-hydroxypicolinic acid (3-HPA) (Kirpekar et al. (2000), RNA 6:296-306; and Tolson & Nicholson (1998), Nucleic Acids Res 26:446-451), 6-aza-2-thiothymine (Gruic-Sovulj et al. (1997), J Biol Chem 272:32084-32091), 2, 4, 6-trihydroxyacetophenone (Patteson et al. (2001), Nucleic Acids Res 29:00), and anthranilic/nicotinic acid (AAINA) mixtures (Zhang & Gross (2000), J Am Soc Mass Spectrom 11:854-865). Among these, only 3-HPA and AA/NA (2.7 mg AA and 1.2 mg NA in 50 μl CH₃CN and 60 μl 50 mM diammonium citrate (DAC)) provided acceptable 74mer signal intensity, and finally, 3-HPA was chosen because it resulted in superior mass resolution and intensity. Moreover, ammonium-loaded cation-exchange beads improved signal intensities dramatically for the 74mer tRNA. More importantly, a ten minute treatment with the NH₄⁺-loaded beads was found to be essential for observing the aminoacyl tRNAs. In addition, H⁺ and N(Bu)₄⁺ forms of the beads were found to produce inferior results.

The final optimized matrix sample preparation routinely resolves 74mer transcripts from 76mer and α-76mer tRNAs. FIG. 3 depicts an experiment where the same tRNA samples that were loaded onto the gel in FIG. 2 were analyzed using MALDI-TOF MS, and shows a transcribed 74mer, a transcribed 76/77mer, a ligated dCA 76mer, and Ala, Trp, and CN-Trp α-76mers. One can see that hydrolysis of the amino acid has been minimal, and confirms that the 76mer observed on the gel in FIG. 2 were produced in the process of running the gel.

Moreover, FIG. 3 shows that the α-76mer peaks are noticeably broader than the 74mer or even the dCA 76mer peak. It is tempting to attribute the change in resolution to contaminants as it seems surprising that aminoacylation, a relatively small change on the scale of a tRNA, would cause such a dramatic change in its behavior in the MALDI-TOF MS. The repeated phenol/chloroform/isoamyl alcohol extractions described in detail in the following examples were crucial to attaining the level of resolution shown in FIG. 3. However, further extraction did not improve the α-76mer signal, so it seems that residual ligation reagents are not limiting the resolution. The loss of resolution observed, hence may be attributable to increased matrix-adduct formation by the aa-76mer relative to the dCA-76mer.

The resolution of aa-76mer from 76mer is aided by the fact that the amino acid is protected on its α-amine with a nitroveratryloxycarbonyl group (NVOC, 241 Da), increasing the mass shift. The inherent error in the system is about 0.1%. This precludes resolving an alanyl tRNA from a glycyl tRNA, but one skilled in the art can still gain ample information about most ligation reactions.

It should be noted that the masses observed are consistent with tRNAs lacking the 5′ phosphorylation expected of a T7 RNA polymerase product. The 5′ phosphate bond has been identified as one of the most labile RNA bonds in MALDI-TOF MS conditions, though RNAs are known to be generally stable in MALDI-TOF MS. See, Kirpekar & Krogh (2001), Rapid Commun Mass Spectrom 15:8-14; Kirpekar et al. (1994), Nucleic Acids Res 22:3866-3870; Knochenmuss et al. (2000), J Mass Spectrom 35:1237-1245; and, Nordhoff et al. (1993), Nucleic Acids Res 21:3347-3357. While this may be cause for concern that any deaminoacylation observed is also a result of the MALDI-TOF MS process, the fact that pure aa-76mer was observed should assuage this concern.

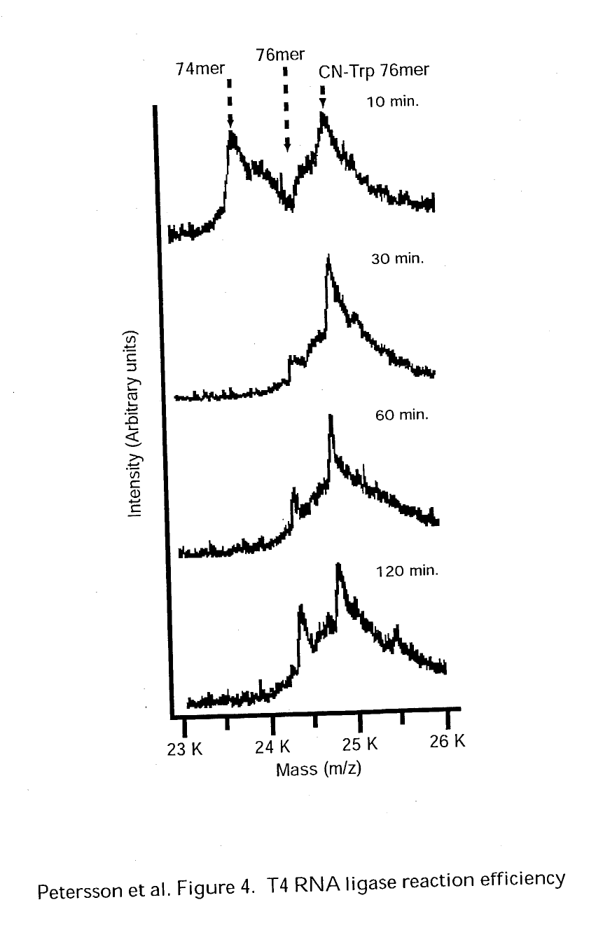

In a valuable application of this methodology, monitoring the T4 ligation reaction by MALDI-TOF MS has shown that the usual two-hour incubation time (Nowak et al. (1998), Methods Enzymol 293:504-529) leads to substantial hydrolysis of the amino acid (FIG. 4). In fact, the reaction is largely complete after 20 minutes, and incubation times longer than 30 minutes are unnecessary. This has held true for a wide variety of both natural and unnatural amino acids, including Ala, Trp, CN-Trp, and two positively charged tyrosine derivatives. While one can get a clear impression of the degree of hydrolysis from mass spectra like those in FIG. 4, it is not possible to quantitate the relative amounts of 76mer and aa-76mer. The decrease in resolution (FIG. 4) for the aa-tRNAs indicates that they ionize differently than non-aminoacyl tRNAs thus making it unreasonable to compare peak intensities.

MALDI-TOF MS can also be used to observe the photocleavage of the NVOC protecting group from the aminoacyl tRNAs. Removing the NVOC protecting group prior to using the tRNA in translation is essential, as the α-amine must be exposed in order for it to be incorporated into the peptide backbone. Time course studies similar to those performed for the ligation reaction can be used to determine optimal photodeprotection conditions. This information is inaccessible through gel electrophoresis.

MALDI-TOF MS has proven useful in evaluating the dCA ligation reaction as well as in examining the deprotection of the α-amines of the aa-76mers. Not only is the MALDI-TOF analysis faster and more material-efficient than gel electrophoresis, it can provide information about the aminoacylation state of the tRNA unobtainable through gels.
a. Materials Used

The synthesis of the pdCpA dinucleotide and its 3′ aminoacylation have been described previously, as have the syntheses of the protected natural and unnatural amino acids which are coupled to the dCA for ligation. See for example, Ellman et al. (1991), Methods Enzymol 202:301-336; and, Nowak et al. (1998), Methods Enzymol 293:504-529. All water used in the enzymatic reactions below was rendered RNase-free by treatment with diethylpyrocarbonate (Sigma-Aldrich, St. Louis, Mo.). The chemicals used in matrix preparation, α-cyano-4-hydroxycinnamic acid (α-CN), 3-hydroxypicolinic acid (3-HPA), picolinic acid (PA), diammonium citrate (DAC), and DOWEX 50WX8-200 100-200 mesh size ion exchange resin, were also purchased from Sigma. The DOWEX beads were exchanged overnight with 1 M NH₄OAc, collected on a frit, and washed twice with 1 M NH₄OAc.
b. Transcription of the 74mer and the 76mer tRNA

The transcription and ligation protocols have been previously described, for example see, Saks et al. (1996), J Biol Chem 271:23169-75. These protocols were used with minor alterations. The tRNA used was THG73, Tetrahymena thermophila tRNA ^{Gln}CUA having a G at position 73. This gene contains an upstream T7 RNA polymerase promoter and downstream restriction sites. Fok I digestion provided the 74mer template and Bsa I digestion gave the 76mer template. The in vitro transcription of linearized cDNA to produce THG73 74mer and 76mer tRNAs was performed with the Ambion T7-MEGAshortscript kit (Austin, Tex.). Transcripts were isolated with a 25:24:1 phenol/CHCl₃/isoamyl alchohol (PCI) extraction. The organic layer was reextracted with water, and a 24:1 CHCl₃/isoamyl alchohol (CI) was performed on the combined aqueous layers. The water layer was then mixed with an equal volume of isopropanol, precipitated overnight at −20° C., pelleted, dried, and redissolved in H₂O. The 76mer tRNA contained a large amount of untemplated 77mer, and hereinafter it is referred to as 76/77mer. There is substantial precedent for the addition of untemplated nucleotides at both the 3′ and 5′ ends of T7 RNA polymerase transcription products. For example see, Helm et al. (1999), RNA 5:618 -621; Kao et al. (1999), RNA 5:1268-1272; Milligan et al. (1987), Nucleic Acids Res 15:8783-8798; and, Pleiss et al. (1998), RNA 4:1313-1317.

c. Ligation of dCA-aa to 74mer tRNA

Prior to ligation, the 74mer tRNAs were heated to 90° C. in a 6.7 mM HEPES (pH 7.5) solution and allowed to cool to 37° C. They were then incubated at 37° C. in 40 μl of a ligation mixture containing 42 mM HEPES (pH 7.5), 10% dimethylsulfoxide (v/v), 4 mM dithiothreitol, 20 mM MgCl₂ 0.2 mg/ml bovine serum albumin (Ambion), 150 μM ATP, 10 μM 74mer tRNA transcript, 300 μM protected dCA-aa, and 2, 000 units/ml T4 RNA ligase (New England Biolabs, Beverly, Mass.). After incubation at 37° C. for 10 to 120 minutes, the reaction mixtures were diluted to 100 μl by adding 8.3 μl 3.0 M NaOAc and 51.7 μl H₂O. They were then extracted against an equal volume of PCI (pH adjusted to 4.5 with NaOAc). The organic layer was re-extracted with 4.2 μl 3.0 M NaOAc and 45.8 μl H₂O. Aqueous layers were combined and extracted again with 150 μl PCI. Two 150 μl CI extractions were performed on the water layer. Finally, the water layer was mixed with 450 μl EtOH and precipitated overnight at −20° C. The sample was pelleted, dried, and resuspended in 1 mM NaOAc to 1.0 μg/μl (DNA quantified by UV absorption at 260 nm).
d. MALDI-TOF Mass Spectrometry

All tRNAs were analyzed on a PerSeptive Biosystems (Framingham, Mass.) Voyager DE PRO MALDI-TOF mass spectrometer operating in linear and positive ion modes. For all experiments the accelerating voltage was held at +25 kV, grid voltage at 92.5%, and guide wire at 0.15%; delay was 500 ns. The nitrogen laser power was set to the minimum level necessary to generate a reasonable signal (except in those experiments in which we attempted to degrade the tRNA). Generally, a two point external calibration was performed, using the M³⁺ (22, 144 Da) and M²⁺ (33,216 Da) peaks of bovine serum albumin (BSA) (PE Biosystems, Foster City, Calif.) in an α-CN matrix (saturated in 2:1H₂O/CH₃CN). For tRNA analyses, the matrix solution consisted of 42 mg 3-HPA, 2 mg PA, and 2 mg DAC dissolved in 500 μl 9:1 H₂O/CH₃CN. A 1.0 μl aliquot of tRNA was exchanged with 2 μl ammonium-loaded cation-exchange beads for ten minutes prior to loading and mixed with 2.0 μL matrix. 0.5 μl of the resulting solution was spotted on the MALDI-TOF sample target and allowed to dry at room temperature. The mass accuracy with external calibration using BSA is estimated to be about 0.1%, or 25 Da for tRNAs of this size. Internal calibrations were performed to eliminate the possibility that mass accuracy was affected by the difference in crystal heights between the α-CN matrix used for calibration and the 3-HPA matrix used with the tRNAs. Apomyoglobin (16, 953 Da) or DNA 40 and 88mers (12, 111 and 27, 210 Da) were used as standards.
e. Gel Electrophoresis

4 μg samples of various tRNA species were resolved on a 20% polyacrylamide (19:1 acrylamide/bis) gel in TBE (10× from BiORad, Hercules, Calif.), 7 M urea, and 0.1 M NaOAc (solution also used to pour gel). The 1.6 mm thick gel was run for 48 hours (1.25 times the amount of time required to run bromophenol blue dye off the gel) at 500 V and stained overnight with Stains-all (Sigma-Aldrich). Procedure adapted from acid/urea gel techniques used by Varshney et al. (Varshney et al., 1991)

1. A method of evaluating the aminoacylated state of tRNAs comprising,
(a) producing aminoacylated tRNAs; and,
(b) using matrix-assisted laser desorption/ionization time-of-flight mass spectrometry (MALDI-TOF MS) to confirm aminoacylation of said tRNA.

2. The method of claim 1, wherein the aminoacylated tRNA is produced by a T4 RNA ligation reaction.

3. The method of claim 1, wherein the tRNA to be aminoacylated is a 74mer, and the aminoacylated tRNA is a 76mer.

4. A method for evaluating the aminoacylation state of a suppressor tRNA that is used to introduce unnatural amino acids into a sidechain or backbone protein by site-directed mutagenesis comprising,
(a) transcribing a desired 74-mer tRNA from a linearized cDNA, wherein said tRNA includes a 74-mer THG733;
(b) ligating the 5′ end of a protected aminoacyl dCA dinucleotide to the 3′ end of said 74mer tRNA; and,
(c) verifying the aminoacylated state of said suppressor tRNA using matrix-assisted laser desorption/ionization time-of-flight mass spectrometry (MALDI-TOF MS).
